(19) United States
(12) Patent Application Publication
(10) Pub. No.: US 2011/0177140 A1
(43) Pub. Date: Jul. 21, 2011

(54) 4-AMIDINO BENZYLAMINES FOR COSMETIC AND/OR DERMATOLOGICAL USE

(76) Inventors: Rainer Voegeli, Bubendorf (CH); Hugo Ziegler, Witterswil (CH); Mathias Gempeler, Pfeffingen (CH)

(21) Appl. No.: 12/674,399

(22) PCT Filed: Aug. 31, 2007
(86) PCT No.: PCT/EP2007/007628

§ 371 Date: Feb. 19, 2010

(51) Int. Cl. A61K 8/64; C07C 311/18; A61K 8/66; A61K 38/48; A61K 38/06; A61K 38/08; A61K 8/65; A61K 38/38; A61K 38/18; A61K 38/30; A61K 38/19; A61K 38/46; A61K 38/44; A61K 8/97; A61K 8/14; A61K 8/11; A61K 8/73; A61Q 19/00; A61P 17/04; A61P 17/00; A61Q 19/08; A61P 17/10; A61Q 17/04

(52) U.S. Cl. 424/401; 564/91; 514/21.91; 424/94.1; 424/94.63; 514/21.9; 514/21.8; 514/17.2; 514/15.2; 514/8.9; 514/9.1; 514/8.5; 514/9.6; 514/9.2; 514/8.4; 514/9.5; 514/8.2; 424/85.1; 514/8.1; 424/94.6; 424/94.4; 424/94.5; 424/755; 424/70.9; 424/70.14; 424/70.13; 424/70.1

(57) ABSTRACT

This invention relates to the use of 4-amidino benzylamine derivatives as cosmetic ingredients and to cosmetic compositions, as well as to non-therapeutic methods for the cosmetic treatment of the skin and the scalp. Said derivatives and compositions can be used as urokinase inhibitors to prevent and restore damage of the epidermal barrier. Barrier abnormalities and disruptions respectively are often the starting point of a dry skin state, of itching, of dandruff and of the perception of sensitive skin. These 4-amidino benzylamine derivatives can be used for topical skin and scalp care applications in form of creams, lotions, gels, shampoos and the like.

### BACKGROUND OF THE INVENTION

Urokinase (uPA), also called urokinase-type plasminogen activator, is a multidomain serine protease (EC 3.4.21.31). uPA is a 411 amino acid residue protein consisting of three domains: the growth factor-like domain (aa 4-43), the kringle domain (aa 47-135) and the catalytic “B” chain (amino acids 144-411) The kringle domain appears to bind heparin. The growth factor-like domain bears some similarity to the structure of epidermal growth factor (EGF), and is thus referred to as an EGF-like domain. uPA is synthesized as a zymogen (pro-uPA or single chain uPA), and is activated by proteolytic cleavage by plasmin between Lys158 and Ile159. The two resulting chains are kept together by a disulfide bond ¹.

uPA is produced by a large variety of cell types such as smooth muscle cells, fibroblasts, endothelial cells, macrophages and tumor cells. It has been implicated as playing a key role in cellular invasion and tissue remodelling ².

In the extracellular matrix uPA is tethered to the cell membrane by its interaction to the specific cell surface receptor uPA receptor (uPAR). The binding interaction is apparently mediated by the EGF-like domain. Cleavage of pro-uPA into active uPA is accelerated when pro-uPA and plasminogen are receptor-bound. Thus, the serine protease plasmin activates pro-uPA, which in turn activates more plasmin by cleaving plasminogen. This positive feedback cycle is apparently limited to the receptor-based proteolysis on the cell surface, since a large excess of protease inhibitors is found in plasma ¹.

The most important endogeneous inhibitors of uPA are the serpins plasminogen activator inhibitor-1 (PAI-1) and plasminogen activator inhibitor-2 (PAI-2), which inhibit the protease activity irreversibly.

A principal substrate for uPA is plasminogen which is converted by cell surface-bound uPA to plasmin. uPA is highly specific to a single peptide linkage in plasminogen. Activated plasmin degrades components of the extracellular matrix (fibrin, fibronectin, laminin, and proteoglycans) and also activates matrix metalloproteases (MMPs) thus promoting the degradation of collagen ^{1, 3, 4}. The activities lead to important processes involving cellular invasion and tissue remodelling and include wound repair, bone remodelling, angiogenesis, tumour invasiveness and spread of metastases ².

Many cell types use uPA as a key initiator of plasmin-mediated proteolytic degradation or modification of extracellular support structures such as extracellular matrix (ECM) and basement membrane (BM). Cells exist, move, and interact with each other in tissues and organs within the physical framework provided by ECM and BM. Movement of cells within ECM or across BM requires local proteolytic degradation or modification of these structures, allowing cells to invade into adjacent areas which were previously unavailable to the cells ⁵.

Accordingly uPA inhibitors have activities against angiogenesis, arthritis, inflammation, invasion, metastasis, osteoporosis and to inhibit growth of tumor ³.

The utility of potent and selective uPA inhibitors is highlighted by the broad range of invasive biological processes mediated by uPA ².

The development of potent and selective inhibitors of uPA is a challenge due to the large number of serine proteases with trypsin-like specificity, including factor VII, factor X and tissue-type plasminogen activator (tPA). Extensive structure-based drug development has provided potent and selective inhibitors of uPA. These generally are arginino mimetics with amidine or guanidine functional groups built onto aromatic or heterocyclic scaffolds ⁶.

Changes in stratum corneum structure, composition and function on different body sites, in different seasons of the year and at different spatial levels of the SC have been of increasing interest to the cosmetic industry over the past few decades. For instance, differences in stratum corneum lipid or NMF (natural moisturizing factor) levels are known to occur on different body sites, lowered levels in the winter months of the year and reductions in their levels occur towards the outer layers of the stratum corneum.

Differences in protease activities and mass levels have also been reported. The epidermis has been shown to express several serine proteases that are involved in multiple activities in skin: epidermal proliferation, differentiation, lipid barrier homeostasis and tissue remodeling. Most importantly proteolysis of stratum corneum corneodesmosomes by serine proteases together with other enzymes is a crucial event prior to desquamation ⁷. The hyperactivity of serine proteases can lead to barrier perturbation due to the degradation of lipid processing enzymes and together with an uncontrolled sustained corneodesmolysis at high pH levels then also deteriorates stratum corneum integrity and cohesion ⁸.

Serine proteases in the stratum corneum may be key markers for underlying and sometimes non-observable skin inflammation. In this respect elevated activity of the plasminogen/plasmin system is thought to impair barrier recovery as protease inhibitors assist barrier recovery ⁹. uPA has been reported to be activated following barrier damage ¹⁰. Increased uPA activity was observed in tape strippings from the cheeks of subjects with dry skin which correlated with increased transepidermal waterloss levels ¹¹. Protease inhibitors especially trypsin-type inhibitors have been reported to reduce dry skin ^{9, 10, 11}.

Kitamura et al. ¹² further demonstrated that plasminogen, that was only located at the basal layer in normal subjects, was expressed in all epidermal cell layers in dry skin. However, Kawai et al. ¹¹ reported that uPA was present in the stratum corneum and this enzyme was the trigger of the activation of the plasminogen system in the stratum corneum. This was elevated in experimentally induced dry skin on back skin of individuals. They further demonstrated that increased uPA activity was present in stratum corneum samples from the cheek in subjects with visibly dry skin and subjects with elevated TEWL levels. If subjects had normal appearing skin and a TEWL of less than approximately 16 g m⁻² h⁻¹, then no activity was found. These findings indicate that an aberration in barrier formation or impaired barrier recovery occurs as a result of elevated TEWL and plasmin activity and that a route for barrier repair would be to use protease inhibitors. Despite this fact the subjects had clinically normal looking skin; the elevated levels on the face may be due to a sub-clinical microinflammatory condition on the face induced by environmental influences.

Repeated barrier disruption induces epidermal hyperplasia and is thought to lead to dry skin ¹⁰.

Surprisingly it was found that uPA-Inhibitors described in WO 01/96286 ⁴ can be used for topical treatment of skin and scalp barrier abnormalities like xerotic skin conditions, itching, dandruff and the perception of sensitive skin.

### DESCRIPTION OF THE INVENTION

The present invention relates to the use of 4-amidino benzylamine derivatives as cosmetic ingredients and for the manufacture of cosmetic and dermatological compositions, as well as in a non-therapeutic process for the cosmetic treatment of the skin. Said 4-amidino benzylamines derivatives are of the general formula (I)

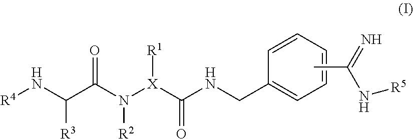

wherein
R¹ represents H, C₁-C₈-alkyl, optionally substituted aryl-C₁-C₄-alkyl, amino-C₁-C₅-alkyl or hydroxy-C₁-C₅-alkyl;
R² represents H or C₁-C₈-alkyl;
R³ represents hydroxy-C₁-C₅-alkyl or C₁-C₈-alkyl;
R⁴ represents H, —SO₂—R, —CO—R, or —COO—R;
R⁵ represents H, OH, —CO—R or —COO—R;
R represents C₁-C₁₆-alkyl, optionally substituted aryl, optionally substituted heteroaryl, optionally substituted aryl-C₁-C₄-alkyl or optionally substituted heteroaryl-C₁-C₄-alkyl and
X represents CH or N.

Preferred are compounds of the general formula (I), wherein the amidino functional group is at position 4 of the phenyl ring and/or wherein

R¹ represents H, C₁-C₈-alkyl, optionally substituted aryl-C₁-C₄-alkyl or amino-C₁—O₅-alkyl;
R² represents H;
R³ represents hydroxy-C₁-C₅-alkyl;
R⁴ represents —SO₂—R;
R⁵ represents H;
R represents optionally substituted aryl-C₁-C₄-alkyl and
X represents CH.

More preferred amidino benzylamine derivatives are benzylsulfonyl-D-Ser-homoPhe-(4-amidino-benzylamide), benzylsulfonyl-D-Ser-Lys-(4-amidino-benzylamide), benzylsulfonyl-D-Ser-Gly-4-amidino-benzylamide and benzylsulfonyl-D-Ser-Ala-4-amidino-benzylamide. All these compounds show potent and highly specific urokinase-inhibiting activity and are described in WO 01/96286 ⁴. These compounds are conveniently used as pure enantiomers.

The term “heteroaryl”, for itself alone or as a structure element for heteroaryl-containing groups, refers to 5 to 11 member aromatic systems composed of one or two rings, wherein 1 to 3 members are heteroatoms, selected among oxygen, sulphur and nitrogen. 1 to 2 benzene rings can be condensed to the heterocycle. Examples thereof are pyridyl, pyrimidinyl, pyridazinyl, pyrazinyl, 1,3,5-triazinyl, quinolinyl, isoquinolinyl, quinoxalinyl, quinazolinyl, phthalazinyl, pyrrolyl, pyrazolinyl, imidazolinyl, 1,2,4-triazolinyl, tetrazolinyl, furyl, thienyl, oxazolinyl, thiazolinyl, isothiazolinyl, benzoxazolyl, benzothienyl, indolyl, benzimidazolyl, indazolyl, benzotriazolyl and benzothiazolyl. The connection can occur either at the hetero moiety or at the benzo moiety and in the π-excess heteroaromates at the nitrogen or any carbon.

Substituents of the optionally substituted aryl- and heteroaryl groups are e.g. halogen, C₁-C₆-alkyl, C₁-C₆-haloalkyl, hydroxy, C₁-C₆-alkoxy, C₁-C₆-haloalkoxy, C₁-C₆-alkenyl, C₂-C₆-alkenyloxy, C₂-C₆-alkinyl, C₃-C₆-alkinyloxy, C₁-C₆-alkoxycarbonyl, CN, OCN, nitro, amino, C₁-C₆-alkylamino, di-C₁-C₆-alkylamino, aminocarbonyl, C₁-C₆-alkylaminocarbonyl, di-C₁-C₆-alkylaminocarbonyl, C₁-C₆-alkylthio, C₁-C₆-alkylsulfoxyl, C₁-C₆-alkylsulfonyl and C₃-C₆-cycloalkyl.

These compounds can be used in cosmetic applications in form of creams, lotions, gels, shampoos and the like, for the treatment of skin and/or scalp barrier abnormalities like xerotic skin conditions, itching, dandruff and/or the perception of sensitive skin.

The topically effective benzylamine derivatives of the present invention can be made available or prepared in any application form desired. Thus, these formulations can be, e.g., an aqueous or anhydrous preparation, an emulsion or micro-emulsion of the water-in-oil (w/o) or oil-in-water (o/w) type, a multiple emulsion, e.g., of the water-in-oil-in-water (w/o/w) type, a gel, a shampoo, a solid, or an aerosol. The formulations of the present invention may be available as, e.g., a powder, a wet patch, a lotion, a cream or an ointment, shampoos and washing formulations, or in any other cosmetically approved form. The effective concentration of the benzylamine derivatives is about 0.001 to 10'000 ppm, preferably 0.1 to 1'000 ppm, related to the total weight of the cosmetic product.

The cosmetically effective benzylamine derivatives of the present invention, which can also be brought into a formulation or a preparation, can be used together with any further, usually applied and topically applicable skin care ingredient. Examples of additional skin care ingredients are derived from plants, algae, microalgae, yeasts, mushrooms, animals and microorganisms, synthetic and semi-synthetic substances,
melatonin, urea, creatinin, dimethylethanolamine and derivatives thereof,
amino acids and derivatives thereof (e.g. serine, glycine, asparagine, cysteine, glutamine, lysine, arginine, aspartic acid, glutamic acid, N-acetylcysteine, citrulline),
proteins, their hydrolyzates and derivatives thereof (e.g. collagen, gelatine, albumin, casein, elastin, keratin, sericin, fibroin, fillagrin),
growth factors and derivatives thereof (e.g. transforming growth factor, insulin-like growth factor, epidermal growth factor, acid and basic fibroblast growth factor, nerve growth factor, keratinocyte growth factor, hepatocyte growth factor, platelet-derived growth factor, granulocyte-macrophage colony stimulating factor, vascular endothelial growth factor),
enzymes and proteases and derivatives thereof (papain, bromelain, subtilisin, superoxide dismutases, lactoperoxidase, phospholipases, transglutaminases),
enzyme inhibitors, protease inhibitors and derivatives thereof (e.g. tranexamic acid, soy bean trypsin inhibitor, Bowman Birk inhibitor, LEKTI, aprotinin, elafin, SLPI, α1-antitrypsin, α1-antichymotrypsin, cholesterol sulfate, leupeptin, chymostatin, tissue inhibitors of metalloproteases, Elhibin®, Colhibin®, compounds of the Pefabloc® series, mustard extract),
co-enzymes and derivatives thereof (e.g. ubiquinon, nicotinamide, nicotinamide adenine dinucleotide, nicotinamide adenine dinucleotide phosphate, coenzyme A, coenzyme B12, flavin adenine dinucleotide, flavin mononucleotide),
peptides such as di-, tri-, tetra-, penta- and hexapeptides and derivatives thereof (e.g. carnosine, H-βAla-Pro-Dab-NHBenzyl, Cu(II)-H-Gly-His-Lys-OH, H-Gly-Leu-Phe-OH, Elaidyl-Lys-Phe-Lys-OH, Palmitoyl-Lys-Val-Lys-OH, H-Lys-Pro-Val-OH, Palmitoyl-Lys-Val-Dab-OH, H-Arg-Ser-Arg-Lys-OH, Palmitoyl-Lys-Val-Dab-Thr-OH, H-Gly-Pro-Arg-Pro-Ala-NH2, Palmitoyl-Lys-Thr-Thr-Lys-Ser-OH, Acetyl-Glu-Glu-Met-Gln-Arg-ArgNH₂),
carbohydrates such as mono-, di-, tri- and oligosaccharides and derivatives thereof (e.g. glucose, fructose, mannose, dihydroxyacetone, erythrulose, saccharose, trehalose, maltoses),
polysaccharides and derivatives thereof (e.g. galactomannans, glucomannans, β-glucans, carrageenans, glycogen, chitosan, lentinans, lichenins, inulins, fucoses, alginates, xyloglucans, dextranes, amyloses, fructanes, xanthans, pullulan),
glycosaminoglycans, their subunits and derivatives thereof (e.g. hyaluronan, chondroitin sulfates, heparin, dermatan sulfates, glucuronic acid, N-acetylglucosamine),
purins, pyrimidines, nucleotides, nucleosides and derivatives thereof (e.g. allantoin, uric acid, adenosine, adenosine monophosphate, adenosine 5′-triphosphate, kinetin),
carboxylic acids and derivatives thereof (e.g. lactic acid, citric acid, glycolic acid, azelaic acid, salicylic acid, lipoic acid, pyrrolidon carboxylic acid, urocanic acids, caffeic acid),
fatty acids and derivatives thereof (e.g. linoleic acid, oleic acid, palmitic acid, conjugated linoleic acid),
lipids and derivatives thereof (e.g. squalane, squalene, monoglycerides, diglycerides, triglycerides, petrolatum, lanolin),
sphingosines, sphingolipids, glycosphingolipids, sulfolipids and derivatives thereof (e.g. phytosphingosines, ceramides, glycoceramides, cerebrosides, gangliobrosides, sulfatides),
phospholipids and derivatives thereof (phosphatidyl choline, phosphatidyl serine, phosphatidyl ethanolamine),
sterols, phytosterols, saponins and derivatives thereof (e.g. cholesterol, sitosterol, stigmasterol, kampesterol, lupeol, glycyrrhizin),
flavonoids and derivatives thereof (e.g. rutin, quercetin, genistein, daidzein, fisetin, myricetin, luteolin, hesperetin, silybin, silymarin, apigenin),
phenols, polyphenols and derivatives thereof (e.g. epigallocatechin, epigallocatechin gallate, resveratrol, nordihydroguaiaretic acid, ellagic acid resorcinol),
terpenes and derivatives thereof (e.g. glycyrrhetinic acid, farnesol, α-bisabolol, β-bisabolol),
alkaloids and derivatives thereof (e.g. caffeine, theophylline, theobromine),
benzofurans and derivatives thereof (e.g. usnic acid),
trace elements (e.g. Zn, Se, Mn) and salts thereof,
polyalcohols and derivatives thereof (e.g. glycerol, propylene glycol, butylene glycol, sorbitol, erythritol, hexanediols, phytantriol),
antimicrobial ingredients, antimicrobial peptides and derivatives thereof (e.g. zinc pyrithione, defensins, cathelicidins, dermcidins, histatin),
UV absorbers and derivatives thereof (e.g. benzoates, anthranilates, salicylates, cinnamates, benzophenones (such as Parsol™ 340), benzimidazoles, benzotriazoles (such as Tinosorb™ M), triazines (such as Tinosorb™ S), polysilicones (such as Parsol™ SLX), titanium oxide, zinc oxide, melanin, avobenzone),
vitamins, provitamins and derivatives thereof (e.g. vitamin A, vitamins of the B series, vitamin C, vitamin D, vitamin E),
retinoids and derivatives thereof (e.g. retinol, retinal, tretinoin, isotretinoin, alitretinoin, etretinate, acitretin, tazarotene, bexarotene),
carotenoids and derivatives thereof (e.g. α-carotene, β-carotene, lycopene, luteine, zeaxanthin, astaxanthin),
chelating agents and derivatives thereof (e.g. EDTA, desferrioxamine, furildioxime),
moisturizers (e.g. glycerol, butylene glycol, sorbitol, urea, N-acetylglucosamine, hyaluronic acid, glycosaminoglycans, amino acids, protein hydrolyzates, collagen, mono-, di, oligo- and polysaccharides, Pentavitin®, Phytaluronate®),
agents regulating the epidermal barrier function (e.g. ceramides, cholesterol, fatty acids, squalane, phytosphingosine, lanolin, lecithin, petrolatum),
skin-revitalizing and regenerating ingredients (e.g. Revitalie-BT, yeast extracts, symphytum extract, ginkgo biloba extract),
skin tightening and anti-wrinkle agents (e.g. centella asiatica, Vialox®, Syn®-Ake, Pefa®-Tight, Matrixyl®, Biopeptide CL, Kollaren PP, elaidyl-Lys-Phe-Lys-OH, H-Arg-Ser-Arg-Lys-OH, Argireline, Collaxyl, Dermican LS 9745),
soothing and anti-inflammatory agents (e.g. camomile extract, panthenol, niacinamide, zinc oxide, aloe vera, calendula extract, licorice extract, hamamelis extract, Sensicalmine, Alistine, H-Lys-Pro-Val-OH),
anti-itching ingredients (e.g. Stimu-Tex®, evening primrose oil),
anti-dandruff ingredients (e.g. allantoin, selenium sulfide, bifonazole, zinc pyrithione),
desquamatory ingredients (e.g. alpha hydroxy acids, beta hydroxy acids),
antioxidants (e.g. superoxide dismutase, ubiquinone, lipoic acid, vitamin E, green tea extract),
sebum regulating and anti acne agents (e.g. Rege-Seb, linoleic acid, pygeum africanum extract, thymus officinalis extract, resorcinol, salicylic acid),
agents regulating stretch marks (e.g. gotu kola extract, Darutosid, Registril),
agents regulating the skin immune system (e.g. arnica extract, Immucell®),
skin lightening agents (e.g. α-arbutin, β-arbutin, kojic acid, magnesium ascorbyl phosphate, licorice extract, Melfade®, Melanostatine, acetyl-Asn-Ser-Leu-Asp-Phe-NH₂),
skin tanning agents (erythrulose, dihydroxyacteone, Melitane PP),
anti-slimming agents (e.g. caffeine, theophylline, guarana extract, Regu®-Fade),
agents regulating the cutaneous microcirculation (e.g. arginine, silybin, silymarin),
agents regulating the primary features of rosacea such as flushing and nontransient erythema (e.g. metronidazole, azelaic acid),
agents regulating couperose and telangiectasia (e.g. silymarin)
antifungal ingredients (e.g. ketokonazole, cyclopyrox, tea tree oil), and mixtures thereof.

Acceptable carriers may generally be used for the manufacture of the cosmetically active composition or formulation of the present invention. Examples of such carriers are, alcohols, polyols, fatty acids, lipids, oils, waxes, thickeners, surfactants, emulsifiers, bulking agents, preservatives, aromas and fragrances as well as staining agents, foam stabilizers and/or silicones.

Carriers to be used in the present invention are in particular glycerine, polyglycerine compounds, ethylene glycol, propylene glycol, polyethylene glycols, polypropylene glycols, ethyl alcohol, isopropyl alcohol, agar gum, gum tragacanth, gum arabic, plant or animal gelatine, methyl cellulose, ethyl cellulose, carboxymethyl cellulose, hydroxymethyl cellulose, hydroxypropyl cellulose, sodium alginate, polyvinyl alcohol, polyvinyl alcohol acetate ester, C₆₋₂₂ fatty alcohols such as cetyl alcohol, C₆₋₂₂ fatty alcohol esters, in particular of stearic acid, palmitic acid, lauric acid and corresponding methyl, ethyl and propyl ester, lanolin, liquid paraffins or natural or synthetic waxes, such as vaseline or beeswax, vegetal oils such as olive oil, coconut oil, soybean oil, castor oil and corresponding hardened oils, hydroxyl-containing compounds modified with polyalkylene oxides, as well as further raw materials known to be incorporated in cosmetic formulations.

For the preparation of a water-in-oil (w/o), oil-in-water (o/w) or water-in-oil-in-water (w/o/w) emulsion or microemulsion, compounds known per se and applied for this purpose are preferably used. For the preparation of the lipid phase, mineral or natural oils or waxes are preferably used. Synthetically manufactured esters of fatty acids with alcohols, such as esters of fatty acids with ethanol, propanol, isopropanol, propylene glycol or glycerine, or esters of fatty alcohols with organic C₃₋₂₀ acids, may be used, too. E.g. esters of myristic acid, palmitic acid, stearic acid, oleic acid, such as propyl myristate, isopropyl palmitate, isopropyl stearate, isopropyl oleate, butyl stearate, hexyl laurate, 2-hexyldecyl stearate, or natural oils, such as jojoba oil, or a mixture thereof are preferred. Preferred silicones are in particular dimethyl polysiloxanes, preferably in cyclic or linear form.

Furthermore, the formulations of the present invention may comprise acids or bases for pH adjustment, e.g. sodium hydroxide, phosphoric acid, citric acid or lactic acid triethanolamine, preferably as a buffer system.

The following examples are intended to explain the present invention more specifically without limiting its scope in any manner.

### EXAMPLES

### Example 1

### Preparation of an Emulsion

Ingredients of phase A are heated to 70° C. and ingredients of phase B to 75° C. Under stirring phase B is poured into phase A. The mixture is cooled to 50° C., homogenized and cooled to 30° C. Then ingredients of phase C and phase D are added. The emulsion is stirred until room temperature is reached.

| Phase | Ingredients | % m/m |
|---|---|---|
| A | Tego Care 450 | 3.00 |
| | Cetearyl alcohol | 2.25 |
| | Glyceryl stearate | 2.25 |
| | Cetiol 868 | 10.00 |
| | Squalane | 5.00 |
| B | Water | 66.99 |
| | Sodium hyaluronate | 5.00 |
| C | Glycerin | 5.00 |
| | Phenonip | 0.50 |
| D | Benzylsulfonyl-D-Ser-Gly-(4-amidino-benzylamide) | 0.01 |

### Example 2

### Preparation of a Cosmetic Gel

Ingredients of phase A are dissolved under stirring. Adjust pH with phase B to 6.0 and then add phase C.

| Phase | Ingredient | % m/m |
|---|---|---|
| A | Water | 92.09 |
| | 1,3-Butanediol | 5.00 |
| | Phenonip | 0.50 |
| | Abil B 8843 | 1.50 |
| | Carboxymethyl Cellulose | 0.15 |
| | Carbopol Ultrez 10 | 0.75 |
| B | NaOH | |
| C | Benzylsulfonyl-D-Ser-Ala-(4-amidino-benzylamide) | 0.01 |

### Example 3

Correlation of TEWL and plasmin and uPA activity in the stratum corneum Ten healthy Caucasian subjects (skin type II-III) participated in the study. All volunteers signed informed consent forms. Before conducting the sequential tape stripping (D-Squame®, CuDerm Corporation, Dallas, USA) on the cheek (9 times) TEWL was measured using an Aquaflux AF103 (Biox Systems, London, UK). The subjects were required not to apply any topical drugs or cosmetics for at least 12 hours before the stratum corneum was sampled. Firstly, 15 minutes before the tape stripping procedure, the skin was carefully cleaned with a cotton pad soaked with distilled water of ambient temperature and allowed to dry. The subjects were acclimated in an environmental room under standard conditions. The skin sites were marked with a surgical marker to ensure that the measurement probes and the tapes were consistently applied to the same area.

Standard D-Squame® disks with a diameter of 2.2 cm and an area of 3.8 cm² were placed on the skin under 225 g/cm² of pressure with a pressure device (CuDerm Corporation, Dallas, USA) for 5 seconds. The interval between the strippings was 20±5 seconds.

The protein content of the tape strippings was quantified by absorption measurements at 850 nm with the infrared densitometer SquameScan™ 850A (Heiland electronic, Wetzlar, Germany). SquameScan™ 850A is especially designed for the application of standard D-Squame® disks. For protein quantification the following equation was used:

C_{protein [μg cm}⁻²_{]}=1.366*Absorption [%]−1.557

Immediately after absorption measurement each tape stripping was transferred into a 1.5 ml Eppendorf tube and extracted for 15 min at 25° C. and 1000 rpm in 750 μl of a buffer composed of 0.1M Tris/HCl and 0.5% Triton X-100 at pH 8.0. The extracts of tape strippings were pooled. To 250 μl of the solutions 1.25 μl of 5 mM fluorogenic urokinase substrate Bz-β-Ala-Gly-Arg-AMC (Pentapharm, Switzerland) and plasmin substrate MeOSuc-Ala-Phe-Lys-AMC (Bachem, Switzerland) dissolved in DMSO were added (final substrate concentration=25 μM). The solutions were mixed at 37° C. and 1000 rpm. The reaction was stopped after 2 hours by adding 100 μl of acetic acid 1% to 100 μl of reaction mixture. The released AMC was quantified by a C18 HPLC gradient elution (80% water/20% acetonitrile/0.07% TFA to 50% water/50% acetonitrile/0.07% TFA). The column used was Symmetry C18, 3.5 μm, 4.6 mm×75 mm (Waters, Milford, USA). The flow rate was 1 ml/min, the injection volume 5 μl and the retention time of AMC 3.5 minutes. The wavelength for emission was 442 nm and for excitation 354 nm.

The data are summarized in Table 1 and FIG. 1. The occurrence of uPA and plasmin activities in the top layers of human stratum corneum allow the conclusion, that these proteases can be inhibited by topically applied inhibitors.

TABLE 1
Determination of the TEWL and uPA and plasmin levels of
the first nine cell layers in the stratum corneum of 10 subjects.

| | TEWL | uPA | Plasmin |
|---|---|---|---|
| Subject | [g m⁻² h⁻¹] | [nU μg⁻¹ protein] | [nU μg⁻¹ protein] |
| 01 | 41.7 | 2.89 | 5.59 |
| 02 | 31.2 | 1.81 | 3.59 |
| 03 | 26.2 | 1.80 | 2.54 |
| 04 | 33.0 | 2.04 | 4.47 |
| 05 | 16.1 | 1.43 | 1.52 |
| 06 | 23.8 | 0.94 | 2.85 |
| 07 | 21.0 | 2.02 | 1.84 |
| 08 | 20.8 | 1.23 | 1.88 |
| 09 | 22.3 | 1.47 | 2.41 |
| 10 | 25.6 | 2.46 | 3.13 |

### REFERENCES

1. Rosenberg, S.; Doyle, M. V. Peptide inhibitors of urokinase receptor activity. U.S. Pat. No. 5,656,726, 1997.
2. Barber, C. G.; Dickinson, R. P. 2-Pyridinylguanidine urokinase inhibitors. EP1044967, Oct. 18, 2000, 2000.
3. Sasaki, T.; Nojima, M. Ozonide compounds with inhibitory activity for urokinase production and angiogenesis. U.S. Pat. No. 6,365,610, 2002.
4. Stürzebecher, J.; Steinmetzer, T.; Künzel, S.; Schweinitz, A. Urokinase Inhibitors. WO 01/96286, Dec. 20, 2001, 2001.
5. Bridges, A.; Schwartz, C. E.; Lottlefield, B. A. Benzothiophenes and thienothiophenes and related compounds useful, for example, as urokinase inhibitors. EP0568289, Nov. 3, 1993, 1993.
6. Deck, L. M.; Vander Jagt, D. L.; Heynekamp, J. J. Isocoumarin-based inhibitors of urokinase-type plasminogen activator. US2006252823, Nov. 9, 2006, 2006.
7. Rawlings, A. V.; Matts, P. J., Stratum Corneum Moisturization at the Molecular Level: An Update in Relation to the Dry Skin Cycle. J Invest Dermatol 2005, 124, (6), 1099-1110.
8. Hachem, J.-P.; Man, M.-Q.; Crumrine, D.; Uchida, Y.; Brown, B. E.; Rogiers, V.; Roseeuw, D.; Feingold, K. R.; Elias, P. M., Sustained Serine Proteases Activity by Prolonged Increase in pH Leads to Degradation of Lipid Processing Enzymes and Profound Alterations of Barrier Function and Stratum Corneum Integrity. J Invest Dermatol 2005, 125, (3), 510-520.
9. Denda, M.; Kitamura, K.; Elias, P. M.; Feingold, K. K., trans-4-(Aminomethyl)cyclohexane Carboxylic Acid (T-AMCHA), an Anti-Fibrinolytic Agent, Accelerates Barrier Recovery and Prevents the Epidermal Hyperplasia Induced by Epidermal Injury in Hairless Mice and Humans. J Invest Dermatol 1997, 109, (1), 84.
10. Katsuta, Y.; Yoshida, Y.; Kawai, E.; Kohno, Y.; Kitamura, K., Urokinase-type plasminogen activator is activated in stratum corneum after barrier disruption. J Dermatol Sci 2003, 32, (1), 55-57.
11. Kawai, E.; Kohno, Y.; Ogawa, K.; Sakuma, K.; Yoshikawa, N.; Aso, D., Can Inorganic Powders Provide Any Biological Benefit in Stratum Corneum, While Residing on Skin Surface. IFSCC Magazine 2002, 5, (4), 269-275.
12. Kitamura, K.; Yamada, K.; Ito, A.; Fukuda, M., Research on the mechanism by which dry skin occurs and the development of an effective compound for its treatment. J Soc Cosmet Chem 1995, 29, 133-145.

1. Use of at least one compound of the following general formula (I) 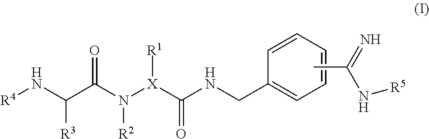
wherein
R¹ represents H, C₁-C₈-alkyl, optionally substituted aryl-C₁-C₄-alkyl, amino-C₁-C₅-alkyl or hydroxy-C₁-C₅-alkyl;
R² represents H or C₁-C₈-alkyl;
R³ represents hydroxy-C₁-C₅-alkyl or C₁-C₈-alkyl;
R⁴ represents H, —SO₂—R, —CO—R, or —COO—R;
R⁵ represents H, OH, —CO—R or —COO—R;
R represents C₁-C₁₆-alkyl, optionally substituted aryl, optionally substituted heteroaryl, optionally substituted aryl-C₁-C₄-alkyl or optionally substituted heteroaryl-C₁-C₄-alkyl and
X represents CH or N
as cosmetic ingredients

2. Use of at least one compound according to claim 1, thereby characterized that the phenyl ring is substituted by the amidino function at position 4.

3. Use of at least one compound according to claim 1, thereby characterized that R¹ represents H, C₁-C₈-alkyl, optionally substituted aryl-C₁-C₄-alkyl or amino-C₁-C₅-alkyl;
R² represents H;
R³ represents hydroxy-C₁-C₅-alkyl;
R⁴ represents —SO₂—R;
R⁵ represents H;
R represents optionally substituted aryl-C₁-C₄-alkyl and
X represents CH.

4. Use of at least one of the compounds
a) benzylsulfonyl-D-Ser-Gly-(4-amidino-benzylamide)
b) benzylsulfonyl-D-Ser-Ala-(4-amidino-benzylamide)
c) benzylsulfonyl-D-Ser-homoPhe-(4-amidino-benzylamide)
d) benzylsulfonyl-D-Ser-Lys-(4-amidino-benzylamide)
according to claim 1.

5. Use of at least one compound according to claim 1, thereby characterized that the compounds are present in the form of dermatologically compatible salts.

6. Use of at least one compound according to claim 1, thereby characterized that the compounds are present as pure enantiomers.

7. Use of at least one compound according to claim 1 for the preparation of a cosmetic composition.

8. Use according to claim 7, thereby characterized that the composition contains the compound(s) of formula (I) in a concentration ranging from 0.001 to 10,000 ppm (m/m), preferably from 0.1 to 1'000 ppm (m/m).

9. A cosmetic composition, in particular a topically applicable cosmetic composition, thereby characterized that it contains
a) at least one compound of the general formula (I) and
b) at least one additional skin care ingredient chosen among amino acids, proteins, protein hydrolyzates, growth factors, enzymes, proteases, enzyme inhibitors, protease inhibitors, co-enzymes, peptides such as di-, tri-, tetra-, penta- and hexapeptides, carbohydrates such as mono-, di-, tri- and oligosaccharides, polysaccharides, glycosaminoglycans, glycosaminoglycan subunits, purins, pyrimidines, nucleotides, nucleosides, carboxylic acids, saturated and unsaturated fatty acids, lipids, sphingosines, sphingolipids, glycosphingolipids, sulfolipids, phospholipids, sterols, phytosterols, saponins, flavonoids, phenols, polyphenols, terpenes, alkaloids, benzofurans, trace elements and salts thereof, polyalcohols, antimicrobial ingredients, antimicrobial peptides, UV absorbers, vitamins, provitamins, retinoids, carotenoids, chelating agents, moisturizers, agents regulating the epidermal barrier function, skin-revitalizing and regenerating ingredients, skin tightening and anti-wrinkle agents, soothing and anti-inflammatory agents, anti-itching ingredients, anti-dandruff ingredients, desquamatory ingredients such as alpha or beta hydroxy carboxylic acids, antioxidants, radical scavengers, UV-quenchers, sebum regulating and anti acne agents, agents regulating stretch marks, agents regulating the skin immune system, skin lightening agents, skin tanning agents, anti-slimming agents, agents regulating the cutaneous microcirculation, agents regulating the primary features of rosacea such as flushing and nontransient erythema, agents regulating couperose and telangiectasia, antimycotic agents, and mixtures thereof.

10. A cosmetic composition according to claim 9, thereby characterized that the additional skin care ingredients are chosen among melatonin, urea, creatinin, dimethylethanolamine, serine, glycine, asparagine, cysteine, glutamine, lysine, arginine, aspartic acid, glutamic acid, N-acetylcysteine, citrulline, collagen, gelatine, albumin, casein, elastin, keratin, sericin, fibroin, fillagrin, transforming growth factor, insulin-like growth factor, epidermal growth factor, acid and basic fibroblast growth factor, nerve growth factor, keratinocyte growth factor, hepatocyte growth factor, platelet-derived growth factor, granulocyte-macrophage colony stimulating factor, vascular endothelial growth factor, papain, bromelain, subtilisin, superoxide dismutases, lactoperoxidase, phospholipases, transglutaminases, tranexamic acid, soy bean trypsin inhibitor, Bowman Birk inhibitor, LEKTI, aprotinin, elafin, SLPI, α1-antitrypsin, α1-antichymotrypsin, cholesterol sulfate, leupeptin, chymostatin, tissue inhibitors of metalloproteases, Elbibin®, Colhibin®, compounds of the Pefabloc® series, mustard extract, ubiquinon, nicotinamide, nicotinamide adenine dinucleotide, nicotinamide adenine dinucleotide phosphate, coenzyme A, coenzyme B12, flavin adenine dinucleotide, flavin mononucleotide, carnosine, H-βAla-Pro-Dab-NHBenzyl, Cu(II)-H-Gly-His-Lys-OH, H-Gly-Leu-Phe-OH, Elaidyl-Lys-Phe-Lys-OH, Palmitoyl-Lys-Val-Lys-OH, H-Lys-Pro-Val-OH, Palmitoyl-Lys-Val-Dab-OH, H-Arg-Ser-Arg-Lys-OH, Palmitoyl-Lys-Val-Dab-Thr-OH, H-Gly-Pro-Arg-Pro-Ala-NH2, Palmitoyl-Lys-Thr-Thr-Lys-Ser-OH, Acetyl-Glu-Glu-Met-Gln-Arg-ArgNH₂, glucose, fructose, mannose, dihydroxyacetone, erythrulose, saccharose, trehalose, maltoses, galactomannans, glucomannans, β-glucans, carrageenans, glycogen, chitosan, lentinans, lichenins, inulins, fucoses, alginates, xyloglucans, dextranes, amyloses, fructanes, xanthans, pullulan, hyaluronan, chondroitin sulfates, heparin, dermatan sulfates, glucuronic acid, N-acetylglucosamine, allantoin, uric acid, adenosine, adenosine monophosphate, adenosine 5′-triphosphate, kinetin, lactic acid, citric acid, glycolic acid, azelaic acid, salicylic acid, lipoic acid, pyrrolidon carboxylic acid, urocanic acids, caffeic acid, linoleic acid, oleic acid, palmitic acid, conjugated linoleic acid, squalane, squalene, monoglycerides, diglycerides, triglycerides, petrolatum, lanolin, phytosphingosines, ceramides, glycoceramides, cerebrosides, gangliobrosides, sulfatides, phosphatidyl choline, phosphatidyl serine, phosphatidyl ethanolamine, cholesterol, sitosterol, stigmasterol, kampesterol, lupeol, glycyrrhizin, rutin, quercetin, genistein, daidzein, fisetin, myricetin, luteolin, hesperetin, silybin, silymarin, apigenin, epigallocatechin, epigallocatechin gallate, resveratrol, nordihydroguaiaretic acid, ellagic acid resorcinol, glycyrrhetinic acid, farnesol, α-bisabolol, β-bisabolol, caffeine, theophylline, theobromine, usnic acid, Zn-, Se-, Mn-salts, glycerol, propylene glycol, butylene glycol, sorbitol, erythritol, hexanediols, phytantriol, zinc pyrithione, defensins, cathelicidins, dermcidins, histatin, benzoates, anthranilates, salicylates, cinnamates, benzophenones, Parsol™ 340, benzimidazoles, benzotriazoles, Tinosorb™ M, triazines, Tinosorb™ S, polysilicones, as Parsol™ SLX, titanium oxide, zinc oxide, melanin, vitamin A, vitamins of the B series, vitamin C, vitamin D, vitamin E, retinol, retinal, tretinoin, isotretinoin, alitretinoin, etretinate, acitretin, tazarotene, bexarotene, α-carotene, β-carotene, lycopene, luteine, zeaxanthin, astaxanthin, EDTA, desferrioxamine, furildioxime, Pentavitin®, Phytaluronate®, Revitalin®-BT, yeast extracts, symphytum extract, ginkgo biloba extract, centella asiatica, Pefa®-Tight, chamomile extract, aloe vera, calendula extract, licorice extract, hamamelis extract, Stimu-Tex®, evening primrose oil, green tea extract, Regu®-Seb, pygeum africanum extract, thymus officinalis extract, gotu kola extract, arnica extract, Immucell®, licorice extract, Melfade®, guarana extract, Regu®-Fade, metronidazole, ketokonazole, cyclopyrox, tea tree oil, and derivatives thereof, and mixtures thereof.

11. A cosmetic composition according to claim 9, thereby characterized that it contains one or several dermatological carriers.

12. A cosmetic composition according to claim 9, thereby characterized that it contains the compounds of formula (I) as a solution, a dispersion, an emulsion, a microemulsion, a nanoemulsion or encapsulated in carriers, preferably as macro-, micro- or nanocapsules, in liposomes or chylomicrons, or enclosed in macro-, micro- or nanoparticles or in microsponges, granulated or absorbed on powdered organic polymers, talc, bentonite and related mineral carriers.

13. A cosmetic composition according to claim 9, thereby characterized that it can be in the form of a solution, an emulsion (oil/water and water/oil), a milk, a lotion, a dispersion, an ointment, gel-forming and viscous surfactants and emulsifying polymers, a pomade, a shampoo, a soap, a gel, a lyophilisate, a powder, a stick, a pencil, a spray, a body oil, a face mask or a patch.

14. Method for the inhibition of proteases, in particular of urokinase, in the skin, thereby characterized that a composition according to claim 9 containing one or several compounds of formula (I) is applied to the skin and/or scalp.

15. Method according to claim 14 thereby characterized that the composition contains benzylsulfonyl-D-Ser-Gly-(4-amidino-benzylamide), benzylsulfonyl-D-Ser-Ala-(4-amidino-benzylamide), benzylsulfonyl-D-Ser-homoPhe-(4-amidino-benzylamide) or benzylsulfonyl-D-Ser-Lys-(4-amidino-benzylamide) or a salt thereof.

16. Non-therapeutic cosmetic method for maintaining, restoring and/or improving the skin hydration, the transepidermal water loss, improving skin and/or scalp barrier abnormalities like xerotic skin conditions, itching, dandruff and/or perception of sensitive skin, thereby characterized that a composition according to claim 9 containing one or several compounds of formula (I) is applied to the skin and/or scalp.

17. Method according to claim 16, thereby characterized that the composition contains benzylsulfonyl-D-Ser-Gly-(4-amidino-benzylamide), benzylsulfonyl-D-Ser-Ala-(4-amidino-benzylamide), benzylsulfonyl-D-Ser-homoPhe-(4-amidino-benzylamide) or benzylsulfonyl-D-Ser-Lys-(4-amidino-benzylamide) or a salt thereof.
